(19) United States
(12) Patent Application Publication
(10) Pub. No.: US 2007/0005034 A1
(43) Pub. Date: Jan. 4, 2007

(54) Ostomy support garment

(76) Inventor: Michael Hansen, Gilleleje (DK)

Correspondence Address: JACOBSON HOLMAN PLLC, 400 SEVENTH STREET N.W., SUITE 600, WASHINGTON, DC, 20004, US

(21) Appl. No.: 10/544,341

(22) PCT Filed: Feb. 3, 2004
(86) PCT No.: PCT/DK04/00074

§ 371 Date: Jun. 7, 2006

(30) Foreign Application Priority Data
Feb. 3, 2003 (DK) PA 2003- 00147

(51) Int. Cl. A61F 5/44

(52) U.S. Cl. 604/345; 604/343

(57) ABSTRACT

An ostomy support garment having a hole for receiving a stoma, said hole having a stabilised edge wherein the edge is provided with a flexible element allowing a deformation of the shape of the hole and providing a snug fit to the stoma ensures an easy application and removal combined with a sufficient support next to the stoma.

### BACKGROUND OF THE INVENTION

1. Field of the Invention

The present invention relates to an ostomy support garment having a hole for receiving a stoma and a method for applying an ostomy support garment to a user.

In connection with surgery for a number of diseases in the gastrointestinal tract a consequence is, in many cases that the colon, the ileum or the urethra has been exposed surgically and the patient is left with an abdominal stoma. Such artificial openings or fistulae cannot be controlled at will and are therefore of necessity incontinent and the effluents or waste products of the body, which are conveyed through these organs, are discharged through the artificial orifice or opening and are collected in a collection bag. Said bag is usually adhered to the skin by means of an adhesive wafer or plate having an inlet opening for accommodating the stoma. Such appliances may be two-piece or one-piece appliances. In both types of appliances, a body side member is attached to the wearer's abdomen, and a receiving member or bag is attached to the body side ostomy member for receiving exudates from the stoma. Said receiving member being attached releasably in case of a two-piece appliance.

In many instances patients having had a surgery resulting in the formation of a stoma, an accompanying condition is formation of a peristomal bulge or hernia, which may complicate the bandaging of the stoma and even require further surgery. Even if further surgery is carried out there is a considerable risk of a permanent condition, which cannot be alleviated.

In such cases, the patient will have to rely on an additional hernia support for a mechanical reposition of the bulge or hernia for reducing the risk of constriction or strangulation calling for urgent surgery and for providing a plane surface around the stoma for application of a collecting appliance in order to secure a proper adherence and sealing. Stomal bulge or hernia supports are commonly known and may e.g. be in the form of a belt of e.g. of leather with buckles, or in the form of a support garment made from an elastic fabric being able to apply a sufficient pressure around the stoma.

In the case of a colostomy and in case the ostomate is normally irrigating, a minor cap or collecting bag may be used which enables the use of a firm support belt or tight compression briefs for providing a sufficient pressure around the stoma. For ileostomists or urostomists this procedure is not practicable due to the constant rather high output from the ileum or bladder and for urostomists it may be critical to provide a free flow from the stoma in order to prevent a build-up of a back pressure, which may destroy the kidneys.

In such cases, it is highly desirable or mandatory to give access to a larger collecting volume, which means that the collecting bag itself will have to be situated outside the pressure establishing belt or briefs and that a passageway through the same has to be established.

Determination of the site for placing the stoma is normally carried out prior to the operation after observing the patient in different postures, e.g. sitting, standing and bending over, finding the less critical area. WO 00/67683 discloses a device for use in the determination and marking of the optimum position of a stoma to be for the patient in question.

As the placing of a stoma is not standardised but depends on the condition and the topography of the abdominal area of the patient, it is not possible to provide a simple selection of standard bulge or hernia supports fitting the majority of patients. The passageways must be tailored according to the actual conditions of the individual patient.

When making a hole in an ostomy support garment it has to be considered that the supporting effect of the missing material has to be provided for in another way and furthermore, it is necessary to stabilise the edge of a hole in order to avoid that it is inadvertently enlarged. At the same time, the effect of the stretching of the support garment and deformation of the shape of the hole when applied has to be taken into consideration as well as the problems associated with providing a sufficiently large hole for allowing an easy passing of an ostomy collection bag during application and removal of the garment and the passing of intestinal contents from the stoma into the bag and at the same time providing a sufficiently snug fit to the stoma to ensure the support next to the stoma.

2. Description of the Related Art

DK Patent Application No. PA 1999 01559 discloses a stomal hernia support compression garment in the form of a pair of compression trousers having a customised hole. The edge of the hole is stabilised by incorporation of a string of nylon sewn with a lockstitch and a zigzag stitch to ensure that the shape of the hole is not changed and furthermore, an enforcement of cotton is sewn using zigzag stitch for stabilising the area around the hole.

U.S. Pat. No. 5,135,520 discloses a variable closure device for an ostomy garment having a pair of criss-cross pocket forming panels configured to lie behind an ostomy device. The criss-cross arrangement of pocket panels are created by finished edges which overlap along their lower ends to define an adjustable, elongated, slanted slot.

It is an object of the present invention to provide a hernia supporting device which provides a hole through which an ostomy bag may be passed, but which is shaped such that the bag is not moved out of the hole by accident. Thus it is an object to provide a hole with an unbroken edge.

Furthermore it is an object to provide a hole which is flexible so as to make it easier for the user to pass the ostomy bag through the hole and such that the hole adapts to the shape of the ostomy device.

Additionally it is an object of the present invention to provide a supporting device that has an inner surface which is as smooth as possible, such that it does not irritate the skin it is pressed against. Thus it is an object of the present invention to provide no or as few seams or overlapping materials as possible.

### SUMMARY OF THE INVENTION

The present invention relates to an ostomy support garment having a hole for receiving a stoma, said hole having a stabilised edge.

Furthermore, the invention relates to a method for applying an ostomy support garment and an ostomy appliance to a user having a stoma and a peristomal bulge or hernia using a two piece ostomy appliance comprising a body side member comprising a hole for receiving the stoma and a receiving bag and an ostomy support garment having a hole for receiving a stoma, said hole having a stabilised edge.

### BRIEF DESCRIPTION OF THE DRAWINGS

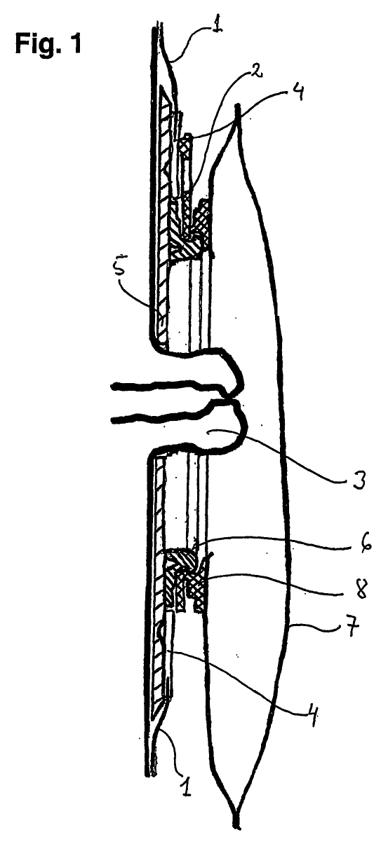

The invention is disclosed more in detail with reference to the drawings in which FIG. 1 shows a sectional view of an embodiment of an ostomy support garment of the invention placed on an ostomate's abdomen together with a two-piece ostomy appliance.

### DETAILED DESCRIPTION OF THE PRESENT INVENTION

The present invention relates to an ostomy support garment having a hole for receiving a stoma, said hole having a stabilised edge wherein the edge is provided with a flexible element allowing a deformation of the shape of the hole and wherein the flexible element is a disc having an internal opening the perimeter of which forms the edge part of the hole.

It may be seen as an advantage of the present invention that the hole (and its edge) is provided in one piece which is both flexible and stable enough to support the ostomy bag. Had the edge of the hole been inflexible it would be harder to pull the ostomy bag through the hole, as the hole could not be temporarily enlarged. Furthermore an unflexible hole could not follow the movement of the user and thus an edge of the hole would apply unwanted pressure to the ostomy bag for some positions of the user e.g. when sitting down or when bending forward.

Thus, the size and the shape of the hole may be temporarily changed for facilitating the application or removal of the garment.

The garment of the invention has a sufficiently rigid edge and provides a snug fit to the stoma to ensure an easy application and removal combined with a sufficient support next to the stoma

It is preferred that such a disc is made from a rigid or semi-rigid polymeric material selected from the group consisting of silicone rubber and polyolefins such as polyethylene, e.g. HD polyethylene, straight or branched polypropylenes and polybutenes and mixtures thereof and copolymers such as EVA, thermoplastic elastomers such as soft PVC rubbers, and polystyrene.

Suitable thermoplastic elastomers are e.g. polyolefin elastomers in the form of copolymers of ethylene and octene. A preferred such elastomer is Engage® 8401 Polyolefin Elastomer from DuPont Dow. Such copolymers may be injection moulded and compression moulded, has a low density reducing the weight of the product and show a suitable stiffness for the purpose of the present invention.

The disc is preferably made from an elastic material having a Shore A-hardness of up to 90, a silicone rubber material having a Shore A-hardness of from 60 to 90 being suitable. A plate of such material provides the following advantages: a) it minimises the risk of fraying of the edge, b) it provides a friction between the base plate of an ostomy appliance and the garment assisting in keeping the garment in place, and c) it provides a relative stiffness replacing the support of the material missing due to the presence of the hole.

The skilled in the art may choose the most suitable material by routine experiments after choosing the desired surface friction, stiffness and resilience of the disc.

The disc suitably has an outer diameter of about 110 mm and the hole suitably a diameter of 65 mm. A hole of this size provides a sufficient support and allows for a reasonably unproblematic passing a collecting bag during application and removal of the ostomy garment.

When using the ostomy support garment of the invention together with a two-piece appliance comprising a body side member and a receiving bag which may be attached via a coupling ring it is preferred that the diameter of the hole is greater than the general outer diameter of the coupling ring of the ostomy body side member in order not to interfere with a safe coupling and also to allow a substitution of the collecting bag without removing the ostomy support garment. Furthermore, the hole is preferably smaller than the diameter of an adhesive wafer of the body side member.

The diameter of the hole may be smaller than the maximum diameter of a coupling system as long as it is large enough not to jeopardize the proper functioning of the coupling system.

The coupling rings may be any system known per se for attaching receiving bags to ostomy body side members and may suitably be matching coupling rings of the type disclosed in WO 94/18919.

Corresponding dimensional considerations apply when using the ostomy support garment of the invention together with a two-piece appliance comprising a body side member and a receiving bag which being attached via matching flanges for adhesive connection e.g. of the type disclosed in U.S. Pat. No. 5,800,415.

When designing ostomy support garments for such cases, care should be taken that the coupling parts be kept essentially plane when the garment is applied in order that the sealing properties of the adhesive coupling are preserved.

A disc of silicone may be moulded directly at the edge of the hole using methods known per se. A suitable method is a RIM-method (Reaction In Mould) in which a two-component mass is cured at room temperature. Room temperature may be seen as temperatures in the interval between 5 and 50 degrees Celsius, such as between 10 and 40 degrees Celsius such as between 15 and 30 degrees Celsius, such as between 18 and 25 degrees Celsius, such as between 19 and 22 degrees Celsius. In the alternative, a disc may e.g. be injection moulded or pressed into the desired shape, preferably in situ. The edge of the hole of the garment is preferably embedded in the silicone material ensuring a stabilisation of the edge of the hole.

The size of the hole may vary in accordance with the stiffness of the disc. For more rigid materials, the size of the hole is suitably larger in order to facilitate the application and detachment of the garment. However, care should be taken not to compromise the support next to the stoma. When using more resilient materials a more snug fit to the stoma is enabled as the edge is softer and thus facilitates the application and detachment of the garment and the collecting device.

It is preferred that the hole in the disc is elongated. When the hole is elongated such as oval or elliptical having its largest dimensions upright (when the user is standing) the hole automatically becomes more circular when the user puts on the garment as it is then stretched horizontally to fit the users torso.

In a second aspect the invention relates to a method for applying an ostomy support garment and an ostomy appliance to a user having a stoma and a peristomal bulge or hernia using a two piece ostomy appliance comprising a body side member comprising a hole for receiving the stoma and a receiving bag and an ostomy support garment having a hole for receiving a stoma, said hole having a stabilised edge, wherein the edge is provided with a flexible element allowing a deformation of the shape of the hole and for providing the supporting effect of the material removed for forming the hole which method comprises locating the stoma and placing the body side member on the users abdomen with the stoma projecting through the hole thereof, applying the collecting bag, aligning the hole of the support garment with the stoma projecting through the hole, pulling the collecting bag through the hole of the garment, and placing and securing the garment on the abdomen of the patient.

In a modification of the second aspect of the invention a one-piece ostomy appliance is used in which case the ostomy appliance is placed on the users abdomen with the stoma projecting into the collecting bag, the hole of the support garment is aligned with the stoma, the collecting bag is pulled through the hole of the garment and the garment is placed and secured on the abdomen of the patient.

### DESCRIPTION OF THE PREFERRED EMBODIMENTS

The invention is now explained more in detail with reference to the drawings showing preferred embodiments of the invention.

Reference is made to FIG. 1 showing a sectional view of an embodiment of an ostomy support garment of the invention placed on an ostomate's abdomen together with a two-piece ostomy appliance.

FIG. 1 shows an ostomy support garment 1 having a hole 2 for receiving a stoma 3, said hole having a stabilised edge in the form of a flexible disc 4. Between the support garment and the patients abdomen is placed an ostomy body side member having an adhesive wafer 5 for adhering to the user's skin. The wafer is provided with a coupling ring 6 for releasable attachment of an ostomy collecting bag 7 having a matching coupling ring 8. As can be seen, the diameter of the hole is greater than the general outer diameter of the coupling ring of the ostomy body side member in order not to interfere with a safe coupling. This allows a substitution of the collecting bag without removing the ostomy support garment.

1. An ostomy support garment having a hole for receiving a stoma, said hole having a stabilised edge wherein the edge is provided with a flexible element allowing a deformation of the shape of the hole and wherein the flexible element is a disc having an internal opening the perimeter of which forms the edge part of the hole.

2. A garment as claimed in claim 1 wherein the edge of the hole of the garment is embedded in the disc.

3. A garment as claimed in claim 1 wherein the flexible element is made from a polymeric material selected from the group consisting of silicone rubber and polyolefins and copolymers thereof.

4. A garment as claimed in claim 1 wherein the opening is elongated.

5. A method for applying an ostomy support garment and an ostomy appliance to a user having a stoma and a peristomal bulge or hernia using a two piece ostomy appliance comprising a body side member comprising a hole for receiving the stoma and a receiving bag and an ostomy support garment having a hole for receiving a stoma, said hole having a stabilised edge, wherein the edge is provided with a flexible element allowing a deformation of the shape of the hole and for providing the supporting effect of the material removed for forming the hole which method comprises locating the stoma and placing the body side member on the users abdomen with the stoma projecting through the hole thereof, applying the collecting bag, aligning the hole of the support garment with the stoma projecting through the hole, pulling the collecting bag through the hole of the garment, and placing and securing the garment on the abdomen of the patient.
